(19) United States
(12) Patent Application Publication
(10) Pub. No.: US 2005/0214931 A1
(43) Pub. Date: Sep. 29, 2005

(54) SYSTEM AND METHOD FOR ANALYZING BIOCHIP

(76) Inventors: Terry B.J. Kuo, Hualien Hsien (TW); Arthur Chiou, Taipei City (TW); Cheng-Yu Wei, Hualien County (TW); Chun-Nan Teng, Kaohsiung City (TW); Kun-Shan Lu, Taipei County (TW)

Correspondence Address: JIANQ CHYUN INTELLECTUAL PROPERTY OFFICE, 7 FLOOR-1, NO. 100, ROOSEVELT ROAD, SECTION 2, TAIPEI, 100, TW

(21) Appl. No.: 10/906,889

(22) Filed: Mar. 11, 2005

(30) Foreign Application Priority Data
Mar. 26, 2004 (TW) 93108234

(51) Int. Cl. C12Q 1/68

(52) U.S. Cl. 435/287.2

(57) ABSTRACT

A system and method for analyzing a biochip are provided. Wherein, a first modulator circuit outputs a first modulated signal with a first frequency. A first light source module output a first light beam based on the first modulated signal. A first optical signal is generated after the first light beam being emitted onto the biochip. An optical sensing module sense the first optical signal and outputting a first sensing signal. A first filter circuit filters the first sensing signal and outputting a first portion sensing signal. A first demodulator circuit demodulates the first portion sensing signal and outputting a first demodulated signal. An analog to digital converter convert the first demodulated signal to a first digital signal. A signal display module receives and display the first digital signal.

### CROSS-REFERENCE TO RELATED APPLICATION

This application claims the priority benefit of Taiwan application serial no. 93108234, filed on Mar. 26, 2004.

### BACKGROUND OF THE INVENTION

1. Field of the Invention

The present invention generally relates to an analyzing system and method, and more particularly to a system and a method for analyzing a biochip.

2. Description of Related Art

Microarray chip or biochip is a newly developed biotechnology and is applied to detect the presence of thousands or tens of thousands of genes. Laser or white light is used to excite and the photo-multiplier tube (PMT) or charge coupled device (CCD) camera is used to detect the fluorescence intensity. The fluorescence intensity reflects the degree of the presence of the genes.

However, the size of the PMT is huge and the PMT requires a significant amount of energy to amplify the tiny fluorescent signal and takes a longer time to scan the entire chip. In addition, when using CCD, low temperature environment is required to prevent the temperature from interfering the tiny fluorescent signal. The above two detecting methods are costly and the CCD cannot be randomly moved due to its big size and its heavy weight. They are also susceptible to external vibration and will cause error.

### SUMMARY OF THE INVENTION

The present invention is directed to a system for analyzing a biochip by using the modulated signals with different frequencies to generate light beam for scanning the biochip and by using the optical sensing module to detect and demodulate in order to obtain scanning and analyzing result.

The present invention is directed to a method for automatically scanning and analyzing a biochip by using modulation technology to significantly reduce external noise.

According to an embodiment of the present invention, the system for analyzing a biochip comprises: a first modulator circuit for outputting a first modulated signal with a first frequency, the first frequency being set as needed; a first light source module, coupled to the first modulator circuit, for outputting a first light beam based on the first modulated signal; a biochip on a path of the first light beam of the first light source module, a first optical signal being generated after the first light beam is emitted onto the biochip; an optical sensing module for sensing the first optical signal and outputting a first sensing signal; a first filter circuit, coupled to the optical sensing module, for filtering the first sensing signal and outputting a first portion sensing signal; a first demodulator circuit, coupled to the first filter circuit, for demodulating the first portion sensing signal and outputting a first demodulated signal; an analog to digital converter circuit, coupled to the first demodulator circuit, for converting the first demodulated signal to a first digital signal; and a signal display module, coupled to an analog to digital converter circuit, for receiving and displaying the first digital signal.

The present invention is directed to a method of automatically scanning and analyzing a biochip. A first modulated signal is provided to a first light source module to generate a first light beam. Next, the first light beam is emitted onto the biochip to generate a first optical signal. Next, the first optical signal is detected and a first sensing signal is output. Next, the first sensing signal is filtered and a first portion sensing signal is output. Thereafter, the first portion sensing signal is modulated and a first demodulated signal is output. Next, the first demodulated signal is converted to a first digital signal, and the first digital signal is output.

According to an embodiment of the present invention, signal modulation technology and the modulated signals with different frequencies are adapted to generate light beam for scanning a biochip, and therefore the external noise is significantly reduced to obtain correct scanning and analysis result.

The above is a brief description of some deficiencies in the prior art and advantages of the present invention. Other features, advantages and embodiments of the invention will be apparent to those skilled in the art from the following description, accompanying drawings and appended claims.

### BRIEF DESCRIPTION OF THE DRAWINGS

FIG. 1 depicts a block diagram of a biochip analyzing system in accordance with an embodiment of the present invention.

FIG. 2 depicts a block diagram of a biochip analyzing system with a plurality of lasers in accordance with another embodiment of the present invention.

FIG. 3A depicts the lateral view of the target biochip and its fluorescent points in accordance with the present invention.

FIG. 3B shows the result on the display after the target biochip of FIG. 3A has been tested in accordance with the present invention.

FIG. 3C is another lateral view of the target biochip and its fluorescent points in accordance with the present invention.

FIG. 3D shows the result on the display after the target biochip of FIG. 3C has been tested in accordance with the present invention.

FIG. 4 shows the modulator circuit and the diode laser driving circuit in accordance with an embodiment of the present invention.

FIG. 5 shows a photo diode and its signal amplifying circuit in accordance with an embodiment of the present invention.

FIG. 6 shows a filter circuit and a demodulator circuit in accordance with an embodiment of the present invention.

### DESCRIPTION OF THE PREFERRED EMBODIMENTS

Generally, a biochip is formed by disposing DNA or protein on a specially chemical-treated glass by contacting or non-contacting way. Then the hybridization will be performed on the biochip and on a tested target. The reacting points of the biochip have to be excited by light with a certain wavelength and can only be sensed by an optical sensing device with high resolution. The following embodiments use laser as the exciting system and the DNA or protein is connected to fluorescent material as marks. By using the laser to excite the fluorescent material to generate fluorescent signal as the base to determine whether the object being analyzed is positive or negative. However, the scope of the present invention is not limited to the fluorescent material as the mark; other exciting light sources or marks can be applied to the present invention.

In addition, this embodiment uses photodiode as a simplified optical sensing device for the biochip analyzing system. This embodiment uses the optical sensing device via modulation with different frequencies so that the same optical sensing device can sense the multi-dimensional fluorescent signal and obtain a high fluorescent signal to noise ratio.

The biochip analyzing system of this embodiment includes the exciting system, the biochip moving system and the fluorescent light sensing system. The new idea that the modulation theory of wireless communication is added to the exciting system and the fluorescent light sensing system so that the exciting system and the optical sensing system are synchronized, which can significantly reduce external noise so as to maximize the strength the fluorescent signal.

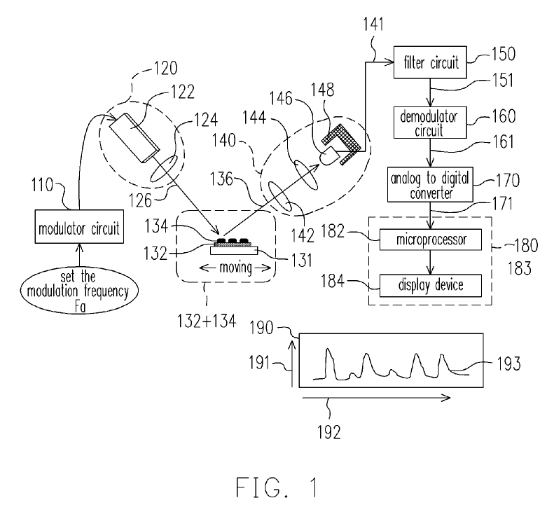

FIG. 1 is a block diagram of a biochip analyzing system in accordance with an embodiment of the present invention. Referring to FIG. 1, in this preferred embodiment, a diode laser 122 is the exciting light source of a light source module 120. A modulator circuit 110 is added to the power supply circuit at the back end of the diode laser 122. The modulator circuit 110 controls the diode laser beam 126 on/off with a frequency Fa and changes the continuous laser beam to an adjustable laser pulse 126. A focusing lens 124 focuses the diode laser beam 126 and the diode laser beam 126 is then emitted onto the surface of the biochip 132+134 at a certain angle. The biochip 132+134 includes a slide 132 and a plurality of fluorescent points 134 (the DNA or the protein marked by the fluorescent material) on the slide 132. The biochip 132+134 is disposed on a moveable device 131. When the biochip 132+134 is moved to a proper place, the fluorescent points 134 with the DNA or the protein with fluorescent material will be excited by the diode laser beam 126 to generate fluorescent signal 136.

The optical sensing module 140 is used for sensing the fluorescent signal 136. The optical sensing module 140 in this embodiment includes a focusing lens 142, an emission filter 144, a photodiode 146 and a circuit 146. In an embodiment, the optical sensing module 140 further includes a metal net 148 (e.g., a copper net) surrounding the photodiode 146. The copper net 148 shields the photodiode from noise interference. The fluorescent signal 136, being focused by the focusing lens 142 and filtered by the emission filter 144, will be emitted onto the sensing portion of the photodiode 146.

The optical sensing module 140 senses the fluorescent signal 136 and sends the result to a filter circuit 150. The filter circuit 150, for example, is a bandpass filter. The filter circuit 150 receives and filters the sensing signal 141 and outputs the sensing signal 151 within a certain frequency band. A demodulator circuit 160 receives the sensing signal 151 and demodulates the sensing signal 151 to the demodulated signal 161. The demodulated signal 161 is converted to the digital signal 171 by an analog to digital converter 170. A signal display module 180 receives and displays the digital signal 171.

The above signal display module 180 for example includes a microprocessor 182 and a display device 184. The microprocessor 182 is coupled to the analog to digital converter circuit 170 for receiving and processing the digital signal 171 and outputting a display signal 183 corresponding to the digital signal. The display device 184 is coupled to the microprocessor 182 for receiving the display signal 183 and displaying an image corresponding to the display signal 183. The display device 184, for example, is a liquid crystal display. The image displayed on the display device is presented, for example, like curve chart 190. The y-axis 191 of the curve chart 190 represents signal intensity and the x-axis 192 represents the time-axis (second) of the biochip 132+134. The curve 193 represents the intensity variation of the fluorescent signal 136 sensed by the optical sensing module 140.

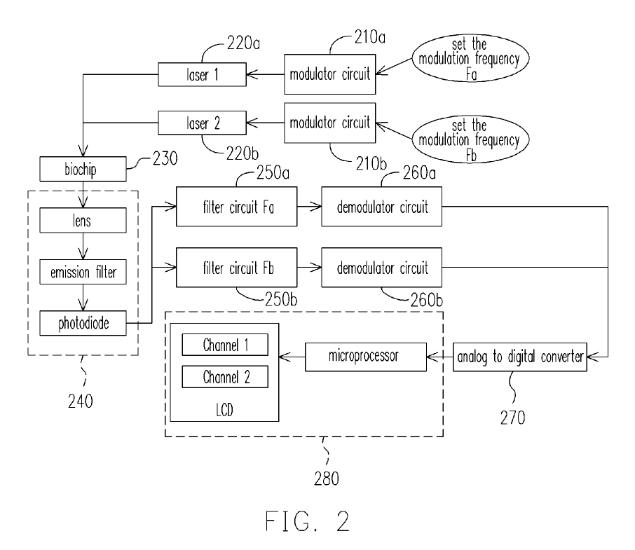

The above embodiment uses only a single laser beam to scan the biochip. However, a plurality of independent laser beams can also be used to scan the biochip. FIG. 2 is a block diagram of a biochip analyzing system with a plurality of lasers in accordance with another embodiment of the present invention. This embodiment is similar to the above embodiment (FIG. 1). The difference is that a plurality of independent laser beams is used to scan the biochip. Referring to FIG. 2, in this embodiment the diode laser is the exciting light source of the light source modules 220a and 220b (for example they are the same as the light source module 120 in FIG. 1). To allow the continuous laser beam to be an adjustable laser pulse, the modulator circuits 210a and 210b (for example they are the same as the modulator circuit 110 in FIG. 1) are added to the power supply circuit at the back end of the light source modules 220a and 220b. The modulator circuits 210a and 210b control the diode laser beams on/off with a frequency Fa and a frequency Fb respectively. The laser beams then are emitted onto the surface of the biochip 230 at different angles. The biochip 230 and the optical sensing module 240, for example, are the same as the biochip 132+134 and the optical sensing module 140.

The optical sensing module 240 sends the sensing result to filter circuits 250a and 250b. The filter circuits 250a and 250b, for example, are bandpass filters with a frequency Fa and bandpass filters with a frequency Fb respectively. The demodulator circuits 260a and 260b receive the sensing signals from the filter circuits 250a and 250b and demodulate the sensing signals to demodulated signals. The demodulated signals are converted to digital signals by an analog to digital converter 270. The signal display module 280 receives and displays the digital signals. The analog to digital converter 270 and the signal display module 280 are the same as the analog to digital converter 170 and the signal display module 180.

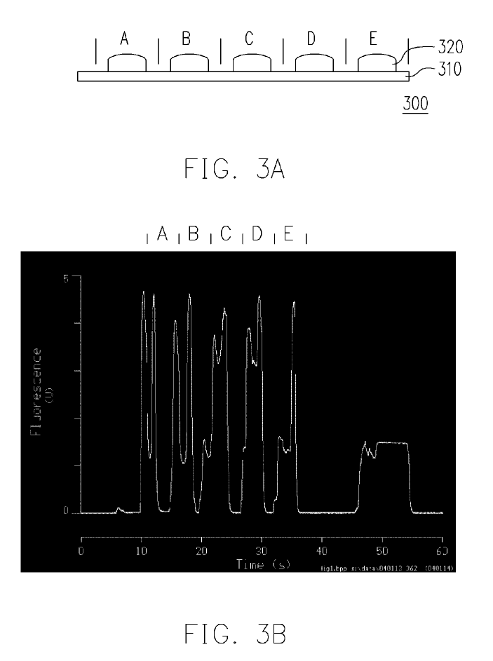

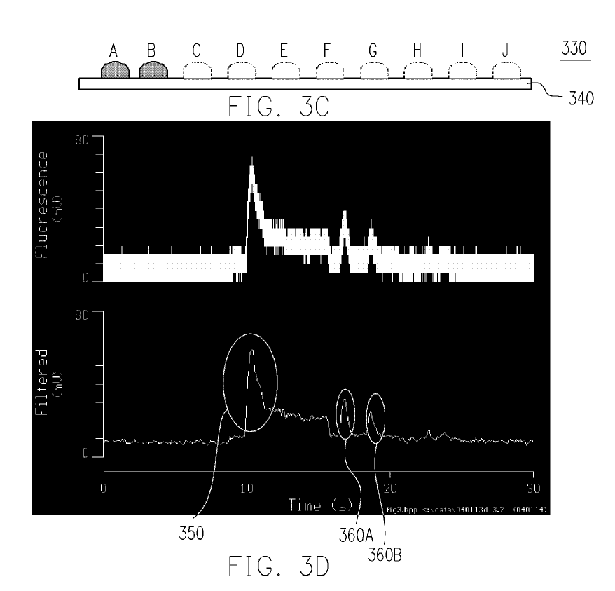

FIG. 3A is a lateral view of the target biochip and its fluorescent points. In FIG. 3A, the slide 310 of the biochip 300 has a plurality of fluorescent points 320 (fluorescent points A˜E). After the biochip 300 is scanned and analyzed by the biochip analyzing system, the display device shows the result as shown in FIG. 3B. As shown in FIG. 3B, the fluorescent points A˜E on the slide 310 are successfully scanned and analyzed and displayed on the display device.

FIG. 3C is a lateral view of the tuberculosis chip after the hybridization. Referring to FIG. 3C, the biochip has a plurality of fluorescent points 340 (fluorescent points A˜J), wherein only fluorescent points A and B have the DNA with the tuberculosis bacteria. After the biochip 330 (the slide 340 and the fluorescent points A˜J) is scanned and analyzed by the biochip analyzing system, the display device shows the result as shown in FIG. 3D. As shown in FIG. 3D, the fluorescent points A and B on the slide 340 are successfully scanned and analyzed and displayed on the display device. The first peak 350 is the reflection of the glass edge. The peaks 360A and 360B are the scanning result of the fluorescent points A and B.

In the embodiment of FIG. 1, the modulator circuit 110 and the diode laser 122 driving circuit can be implemented as shown in FIG. 4. FIG. 4 shows the modulator circuit and the diode laser driving circuit in accordance with another preferred embodiment of the present invention. The first terminal of the capacitor C1 is coupled to the negative input terminal of the OP amplifier OP1 and the second terminal of the capacitor C1 is grounded. In this embodiment, the capacitance of the capacitor C1 is, for example, 15 pF. The first terminal of the resistor R1 is coupled to the negative input terminal of the OP amplifier OP1 and the second terminal of the resistor R1 is coupled to the output terminal of the OP amplifier OP1. The resistor R1 is an adjustable resistor; the resistance ranges from 22KΩ to 122KΩ.

The first terminal of the resistor R2 is coupled to the output terminal of the OP amplifier OP1 and the second terminal of the resistor R2 is coupled to the positive input terminal of the OP amplifier OP1. The first terminal of the resistor R3 is coupled to the positive input terminal of the OP amplifier OP1 and the second terminal of the resistor R3 is grounded. The resistance of the resistors R2 and R3 is 100KΩ and 66KΩ respectively. The resistor R4 is an adjustable resistor with a first, a second, and a middle terminal. The resistance of the resistor R4 can be adjusted by adjusting the resistance between the middle and the first terminals and between the middle and the second terminals. The maximum resistance of the resistor R4 is 10KΩ; the second terminal of the resistor R4 is grounded. The anode of the diode D1 is coupled to the output terminal of the OP amplifier OP1 and the cathode of the diode D1 is coupled to the first terminal of the resistor R4.

The positive input terminal of the OP amplifier OP2 is coupled to the middle terminal of the resistor R4. The first terminal of the resistor R5 is coupled to the negative input terminal of the OP amplifier OP2 and the second terminal of the resistor R5 is grounded. In this embodiment, the resistance of the resistor R5 is 5.6Ω. The base of the transistor NPN is coupled to the output terminal of the OP amplifier OP2; the emitter is coupled to the negative input terminal of the OP amplifier OP2; the collector is coupled to the cathode of the laser diode LD. The anode of the laser diode LD is coupled to the system voltage VCC (e.g., 5V). The laser beam obtained by driving the laser diode LD is the diode laser beam 126 of FIG. 1.

In addition, the photodiode and its circuit 146 of FIG. 1 can be implemented as the photodiode and its circuit shown in FIG. 5. FIG. 5 shows a photodiode and its signal amplifying circuit in accordance with a preferred embodiment of the present invention. The anode of the photodiode PD is grounded; the cathode is coupled to the negative input terminal of the OP amplifier OP3. The positive input terminal of the OP amplifier OP3 is grounded. The first terminal of the capacitor C2 is coupled to the negative input terminal of the OP amplifier OP3; the second terminal of the capacitor C2 is coupled to the output terminal of the OP amplifier OP3. The first terminal of the resistor R6 is coupled to the negative input terminal of the OP amplifier OP3; the second terminal of the resistor R6 is coupled to the output terminal of the OP amplifier OP3. The resistance of the resistor R6 is 20MΩ. The capacitance of the capacitor C2 is 10 pF.

The first terminal of the capacitor C3 is coupled to the output terminal of the OP amplifier OP3; the second terminal of the capacitor C3 is coupled to the positive input terminal of the OP amplifier OP4. The first terminal of the resistor R7 is coupled to the positive input terminal of the OP amplifier OP4; the second terminal of the resistor R7 is grounded. The resistance of the resistor R7 is 10KΩ. The capacitance of the capacitor C3 is 0.47 μF. The resistor R8 is an adjustable resistor with a first, a second, and a middle terminal. The resistance of the resistor R8 can be adjusted by adjusting the resistance between the middle and the first terminals and between the middle and the second terminals. The first terminal of the resistor R8 is grounded; the second terminal is coupled to the output terminal of the OP amplifier OP4; the middle terminal is coupled to the negative input terminal of the OP amplifier OP4. The maximum resistance of the resistor R4 is 10KΩ. The output terminal of the OP amplifier OP4 is coupled to the filter circuit 150 of FIG. 1 and outputs the sensing signal 141.

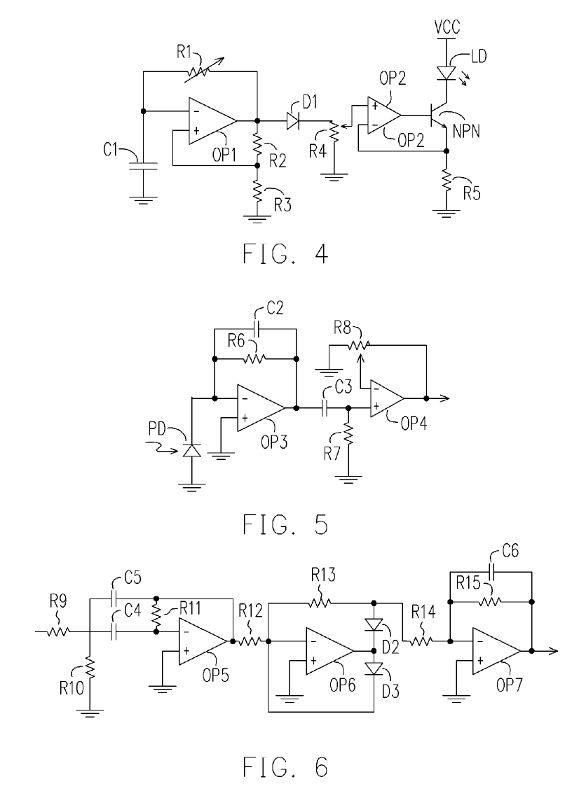

The filter circuit 150 and the demodulator circuit 160 of FIG. 1 can be implemented as shown in FIG. 6. FIG. 6 shows a filter circuit and a demodulator circuit in accordance with an embodiment of the present invention. The first terminal of the resistor R9 is coupled to the output terminal of the photodiode and its circuit 146; the second terminal of the resistor R9 is coupled to the first terminal of the capacitor C4. The second terminal of the capacitor C4 is coupled to the negative input terminal of the OP amplifier OP5. The positive input terminal of the OP amplifier OP5 is grounded. The resistance of the resistor R9 is 2KΩ. The capacitance of the capacitor C4 is 0.015 μF.

The first terminal of the capacitor C5 is coupled to the first terminal of the capacitor C4; the second terminal of the capacitor C5 is coupled to the output terminal of the OP amplifier OP5. The first terminal of the resistor R10 is coupled to the first terminal of the capacitor C4; the second terminal of the resistor R10 is grounded. The first terminal of the resistor R11 is coupled to the output terminal of the OP amplifier OP5; the second terminal of the resistor R11 is coupled to the negative input terminal of the OP amplifier OP5. The resistance of the resistors R10 and R11 are 510Ωand 270KΩ respectively. The capacitance of the capacitor C5 is 0.015 μF.

The first terminal of the resistor R12 is coupled to the output terminal of the OP amplifier OP5; the second terminal of the resistor R12 is coupled to the negative input terminal of the OP amplifier OP6. The positive input terminal of the OP amplifier OP6 is grounded. The first terminal of the resistor R13 is coupled to the negative input terminal of the OP amplifier OP6; the second terminal of the resistor R13 is coupled to the anode of the diode D2. The cathode of the diode D2 is coupled to the output terminal of the OP amplifier OP6. The anode of the diode D3 is coupled to the output terminal of the OP amplifier OP6; the cathode of the diode D2 is coupled to the negative input terminal of the OP amplifier OP6. The resistance of the resistors R12 and R13 are 5KΩ and 100KΩ respectively.

The first terminal of the resistor R14 is coupled to the anode of the diode D2; the second terminal of the resistor R14 is coupled to the negative input terminal of the OP amplifier OP7. The first terminal of the resistor R15 is coupled to the negative input terminal of the OP amplifier OP7; the second terminal of the resistor R15 is coupled to the output terminal of the OP amplifier OP7. The first terminal of the capacitor C6 is coupled to the negative input terminal of the OP amplifier OP7; the second terminal of the capacitor C6 is coupled to the output terminal of the OP amplifier OP7. The positive input terminal of the OP amplifier OP7 is grounded. The output terminal of the OP amplifier OP7 is coupled to the analog to digital converter 170 of FIG. 1 and outputs the demodulated signal 161. The resistance of the resistors R14 and R15 are 100KΩ and 220KΩ respectively. The capacitance of the capacitor C5 is 0.47 μF.

The above description provides a full and complete description of the preferred embodiments of the present invention. Various modifications, alternate construction, and equivalent may be made by those skilled in the art without changing the scope or spirit of the invention. Accordingly, the above description and illustrations should not be construed as limiting the scope of the invention which is defined by the following claims.

1. A system for analyzing a biochip, comprising:
a first modulator circuit, for outputting a first modulated signal with a first frequency, said first frequency being set as needed;
a first light source module, coupled to said first modulator circuit, for outputting a first light beam based on said first modulated signal;
a biochip, on a path of said first light beam of said first light source module, a first optical signal being generated after said first light beam being emitted onto said biochip;
an optical sensing module, for sensing said first optical signal and outputting a first sensing signal;
a first filter circuit, coupled to said optical sensing module, for filtering said first sensing signal and outputting a first portion sensing signal;
a first demodulator circuit, coupled to said first filter circuit, for demodulating said first portion sensing signal and outputting a first demodulated signal;
an analog to digital converter circuit, coupled to said first demodulator circuit, for converting said first demodulated signal to a first digital signal; and
a signal display module, coupled to said analog to digital converter circuit, for receiving and displaying said first digital signal.

2. The system of claim 1, further comprising:
a second modulator circuit, for outputting a second modulated signal with a second frequency, said second frequency being set as needed;
a second light source module, coupled to said second modulator circuit, for outputting a second light beam based on said second modulated signal, a second optical signal being generated after said second light beam being emitted onto said biochip, said optical sensing module sensing said second optical signal and outputting a second sensing signal;
a second filter circuit, coupled to said optical sensing module, for filtering said second sensing signal and outputting a second portion sensing signal;
a second demodulator circuit, coupled to said second filter circuit, for demodulating said second portion sensing signal and outputting a second demodulated signal;
wherein said analog to digital converter circuit converts said first demodulated signal and said second demodulated signal to a first digital signal and a second digital signal respectively, and said signal display module receives and displays said first digital signal and said second signal.

3. The system of claim 1, wherein said first light source module is a laser module and said first light beam is a first laser beam.

4. The system of claim 3, wherein said laser module comprises:
a laser generator, coupled to said first demodulator circuit, for receiving said first modulated signal and outputting a first laser beam corresponding to said first modulated signal; and
a first lens, for focusing said first laser beam.

5. The system of claim 1, wherein said optical sensing module comprises:
a second lens for focusing said first optical signal;
an emission filter for filtering said first optical signal; and
a photodiode for sensing said first optical signal and generating said first sensing signal corresponding to said first optical signal.

6. The system of claim 5, wherein said optical sensing module further comprises a metal net surrounding said photodiode.

7. The system of claim 6, wherein said metal net is a copper net.

8. The system of claim 1, wherein said signal display module comprises:
a microprocessor, coupled to said analog to digital converter circuit, for receiving and processing said first digital signal and outputting a display signal corresponding to said first digital signal; and
a display device, coupled to said microprocessor, for receiving said display signal and displaying an image corresponding to said display signal.

9. The system of claim 8, wherein said display device is a liquid crystal display.

10. The system of claim 1, wherein said biochip is disposed on a movable device allowing said biochip being moveable.

11. The system of claim 1, wherein said biochip is a gene chip.

12. The system of claim 1, wherein said biochip is a protein chip.

13. A method for automatically scanning and analyzing a biochip, comprising:
providing a first modulated signal to a first light source module to generate a first light beam;
emitting said first light beam onto said biochip to generate a first optical signal;
sensing said first optical signal and outputting a first sensing signal;
filtering said first sensing signal and outputting a first portion sensing signal;
demodulating said first portion sensing signal and outputting a first demodulated signal;
converting said first demodulated signal to a first digital signal; and
outputting said first digital signal.

14. The method of claim 13, further comprising:
providing a second modulated signal to a second light source module to generate a second light beam;
emitting said second light beam onto said biochip to generate a second optical signal;
sensing said second optical signal and outputting a second sensing signal;
filtering said second sensing signal and outputting a second portion sensing signal;
demodulating said second portion sensing signal and outputting a second demodulated signal;
converting said second demodulated signal to a second digital signal; and
outputting said second digital signal.

15. The method of claim 14, wherein said first light beam is generated based on said first modulated signal and said first modulated signal has a first frequency; said second light beam is generated based on said second modulated signal and said second modulated signal has a second frequency.

16. The method of claim 14, wherein said first light beam and said second light beam are emitted to different positions of said biochip respectively.

17. The method of claim 14, wherein each of said first light beam and said second light beam is a laser beam.

18. The method of claim 13, wherein said first optical signal is a fluorescent signal.

19. The method of claim 14, wherein said second optical signal is a fluorescent signal.
